(19) United States
(12) Patent Application Publication
(10) Pub. No.: US 2012/0047589 A1
(43) Pub. Date: Feb. 23, 2012

(54) METHOD OF DELIVERY OF NUCLEIC ACIDS TO A DEVELOPING EMBRYO

(76) Inventors: Theresa E. Gratsch, Ann Arbor, MI (US); J. Matthew Velkey, Ann Arbor, MI (US); Lisa S. D. Emmett, Ann Arbor, MI (US); K. Sue O'Shea, Ann Arbor, MI (US)

(21) Appl. No.: 13/283,342

(22) Filed: Oct. 27, 2011

Related U.S. Application Data
(63) Continuation of application No. 12/249,452, filed on Oct. 10, 2008, now abandoned.
Application No. 12/249,452 is a continuation of application No. 12/028,356, filed on Feb. 8, 2008, now abandoned.
(60) Provisional application No. 60/900,185, filed on Feb. 8, 2007.

(51) Int. Cl. C12N 15/87; A61B 17/435; A61M 1/00; A61M 25/00; A61K 48/00; A61M 5/00

(52) U.S. Cl. 800/21; 514/44 R; 604/506; 604/8; 604/500; 600/34

(57) ABSTRACT

Methods to introduce genetic material, such as DNA, to embryos, are disclosed. In some embodiments, the method involves preparing the pregnant mother to receive the genetic material into a blood transport vessel which passes to the embryo, avoiding a maternal capillary bed and introducing the material under low pressure so as not to kill the pregnant animal. The effectiveness of the method is such that the nucleic acid has been expressed in all the cells of the embryo and in the postnatal mouse, including the primordial germ cells, thus making the nucleic acid germ line heritable.

The present application is a continuation of U.S. patent application Ser. No. 12/249,452 filed Oct. 10, 2008, which is a continuation of U.S. patent application Ser. No. 12/028,356 filed Feb. 8, 2008, which claims priority to U.S. Provisional Patent Application Ser. No. 60/900,185, filed Feb. 8, 2007, the disclosures of which are herein incorporated by reference in their entirety.

### STATEMENT OF GOVERNMENT INTEREST

This invention was made with government support under GRANTS-NS-39438 and RR-23187, awarded by the National Institutes of Health. The government has certain rights in the invention.

### FIELD OF THE INVENTION

This disclosure provides methods of altering the gene expression of an embryo by delivering nucleic acid to the embryo by administering the nucleic acid into the pregnant mother.

### SUMMARY

Disclosed in this specification are processes and systems to modify the genes or gene expression of a mammalian embryo. In some embodiments, a method comprises the steps of preparing a pregnant animal carrying the embryo for at least one injection, inserting a hollow device into a blood transport vessel of the pregnant animal, and introducing genetic material into the blood transport vessel through the hollow device. It is further disclosed that the step of preparing the pregnant animal to receive the at least one injection further comprises the step of dilating the blood transport vessel and that the step of dilating the blood transport vessel may be done, for example, by warming the pregnant animal and topical alcohol application.

The specification further discloses embodiments that involve immobilizing the blood transport vessel of the pregnant animal and that such immobilization may include one or more of: anesthetizing the pregnant animal, sufficiently securing the pregnant animal to permit insertion of the hollow device into the blood transport vessel, and having the pregnant animal immobilize the blood transport vessel.

Also disclosed are methods and systems that employ a hollow device. In some embodiments, the device includes, but is not limited to a needle and shunt. In some embodiments, the needle may be a butterfly needle. In some embodiments, the needle may be 23 or 26 gauge needle.

In some embodiments, the blood transport vessel includes, but is not limited to, blood transport vessels which transport blood from the point of insertion of a hollow device to the embryo, avoiding reaching a capillary bed. In some embodiments, the blood transport vessel is on or more of uterine arteries, tail veins and the functional equivalent of tail veins in animals without tails.

In some embodiments, the introduction of the genetic material is done after implantation of the embryo into a uterine wall and prior to establishment of the fetal circulation and more preferably done as the embryo begins gastrulation and prior to establishment of the fetal circulation. For example, where the pregnant animal is a mouse, the introduction of the genetic material may be done at about E6.5 of embryonic development, where the morning of finding a vaginal plug is considered E0.5.

The introduction pressure is also disclosed and is sufficiently low so as not to kill the pregnant animal. Exemplary, sufficient pressures disclosed are when the introduction pressure is below about the pressure required to pass 300 microliters of Ringer's solution at 23° C. through a 23 gauge needle in 10 seconds; or below about the pressure required to pass 300 microliters of Ringer's solution at 23° C. through a 23 gauge needle in 15 seconds; or below about the pressure required to pass 300 microliters of Ringer's solution at 23° C. through a 23 gauge needle in 20 seconds; or below about the pressure required to pass 300 microliters of Ringer's solution at 23° C. through a 23 gauge needle in 25 seconds; or is below about the pressure required to pass 300 microliters of Ringer's solution at 23° C. through a 23 gauge needle in 30 seconds; or the introduction of the genetic material is done through a drip into the blood transport vessel. In some embodiments, the introduction of the genetic material is done in a discontinuous manner.

In some embodiments, the introduction of the genetic material is present in a carrier solution with the carrier solution preferably of sufficient polarity to keep the genetic material dispersed. Ringer's solution is disclosed as an exemplary carrier solution.

The present invention is not limited by the nature of the genetic material that is delivered. In some embodiments, the genetic material is DNA, naked DNA, and all variants of RNA. In some embodiments, the DNA is in the form of an expression vector encoding any desired sequence of interest. Sequences of interest include, but are not limited to, sequences encoding proteins, peptides, siRNA, miRNA, rRNA, and the like. The expression vector may comprise a promoter sequence to permit tissue-specific or inducible expression of the nucleic acid of interest.

It is also disclosed that because this method creates gene alterations in all cells, including the primordial germ cells, that this method is useful in creating transgenic animals in which the genetic material is germ line heritable; that is that the embryos born will be able to pass the alteration to their offspring. It is also disclosed that at the embryo level this method provides a much more efficient way to study the impact of gene knock-down and expression on the developing embryo, provide a much more efficient way to produce transgenic animals, and provide a much more efficient way to permanently alter genes in the germ line. The methods may be used in both research and therapeutic settings. In either setting, for example, the methods may be used to control gene expression during development of an embryo to control one or more biological pathways or processes.

### DESCRIPTION OF FIGURES

FIG. 1 depicts one sample construct which can be injected into the blood transport vessel.

### BACKGROUND

It presently takes an exceedingly long time to make transgenic animals (ones in which their genetic constitution is altered). For example, it takes one year to make a transgenic mouse using current techniques. One method of making a transgenic animal requires introducing the DNA containing the genetic alteration into fertilized eggs, then letting resulting embryos be born, breeding those mice (generation F0) to determine which have incorporated the change into the germ line (most will not). An alternative technique is to introduce the alteration into embryonic stem cells (ESCs) and use those stem cells to make embryos, but the breeding is the same. The disclosed technique short circuits this process by not needing to inject into fertilized eggs which requires molecular technology and specialized equipment or ESCs.

With the sequencing of the genome, there has been tremendous interest in teasing out the function of every gene. In the mouse, gene targeting using homologous recombination in embryonic stem cells (ESC) has provided a unique opportunity to probe gene function in development (Capecchi, 1989), and a number of powerful new techniques have been developed to target genes in temporal or tissue specific ways. Unfortunately these are time consuming and often require development of multiple strains of mice, which then must be mated to obtain the desired cell-type specific gene targeting.

Much time and effort has been spent on gene transduction (using viral vectors, or non-naked DNA) of the embryo. In utero gene targeting/therapy has been proposed as a method to treat a number of diseases that affect the developing embryo (Coutelle et al., 1995), and may ultimately be the most effective means to treat genetic defects. Much research examining plasmid DNA delivery has been carried out for fetal “gene therapy” including: direct injection of the fetus (Baldwin et al., 1997; Larson et al., 1997; Gaensler et al., 1999), injection into the placenta or umbilical cord (Papaioannou, 1990; Turkay et al., 1999; Mitchell et al., 2000), injection into the amniotic cavity (Douar et al., 1997; Schatchner et al., 1999; Mitchell et al., 2000) or the yolk sac (Schachtner et al., 1999) generally resulting in localized transduction of the embryo.

While intravascular delivery of naked DNA is increasingly recognized as a preferred route to deliver nucleic acids to target tissues (Hodges and Scheule, 2003) because of its simplicity and effectiveness and because high levels of transgene expression can be achieved and sustained (e.g., Hagstrom et al., 2004), successful techniques for the introduction of naked DNA into the embryo via intravascular injection of the pregnant mother has not been reported.

Lewis et al. (Nature Genetics, volume 32, 2002) disclose use of high pressure tail vein injections into postnatal mice. This references does not disclose the use of tail vein injection in a pregnant animal with a placenta and embryo.

The following are references useful for this field and referred throughout the specification, and are herein incorporated by reference in their entireties.
Baldwin H S, Mickanin C, Buck C. 1997. Adenovirus-mediated gene transfer during initial organogenesis in the mammalian embryo is promoter-dependent and tissue-specific. Gene Ther 4: 1142-1149.
Capecchi M R. 1989. Altering the genome by homologous recombination. Science 244: 1288-1292.
Coutelle C, Douar A M, Colledge W H, Froster U. 1995. The challenge of fetal gene therapy. Nature Med 1: 864-865.
Douar A M, Adebakin S, Themis M, Pavirani A, Cook T, Coutelle C. 1997. Foetal gene delivery in mice by intra-amniotic administration of retroviral producer cells and adenovirus. Gene Ther 4: 883-390.
Gaensler K M L, Tu G, Bruch S, Liggitt D, Lipshutz G S, Metkus A, Harrison M, Heath T D, Debs R J. 1999. Fetal gene transfer by transuterine injection of cationic liposome-DNA complexes. Nature Biotech 17: 1188-1192.
Hagstrom J E, Hegge J, Zhang G, Noble M, Budker V, Lewis D L, Herweijer H, Wolff J A. 2004. A facile nonviral method for delivering genes and siRNAs to skeletal muscle of mammalian limbs. Mol Ther 10: 386-398.
Hodges B L, Scheule R K. 2003. Hydrodynamic delivery of DNA. Expert Opin Biol Ther 3: 911-918.
Larson J E, Morrow S L, Happel L, Sharp J F, Cohen J C. 1997. Reversal of cystic fibrosis phenotype in mice by gene therapy in utero. Lancet 349: 619-620.
Lewis D L, Hagstrom J E, Loomis A G, Wolff J A, Herweijer H. 2002. Efficient delivery of siRNA for inhibition of gene expression in postnatal mice. Nature Genet 32: 107-108.
Mitchell M, Jerebtsova M, Batshaw M L, Newman K, Ye X. 2000. Long-term gene transfer to mouse fetuses with recombinant adenovirus and adeno-associated virus (AAV) vectors. Gene Ther 7: 1986-1992.
Papaioannou V E. 1990. In utero manipulation. In: Copp A J, Cockroft D L (eds). Postimplantation Mammmalian Embryos. IRL Press at Oxford University Press: Oxford, pp 61-80.
Schachtner S K, Buck C A, Bergelson J M, Baldwin H S. 1999. Temporally regulated expression patterns following in utero adenovirus-mediated gene transfer. Gene Ther 6: 1249-1257.
Turkay A, Saunders T L, Kurachi K. 1999. Intrauterine gene transfer: gestational stage-specific gene delivery in mice. Gene Ther 6: 1685-1694.

### DETAILED DESCRIPTION

The following description provides exemplary embodiments of the present invention. The present invention is not limited to these particular embodiments.

It was discovered during the development of embodiments of the present invention that when genetic material is injected into the pregnant mother at the right time and in the right conditions, for example, at pressures lower than those used in the art, the genetic material is expressed in all the cells of embryo, including the primordial germ cells. As will be explained in greater detail later, the method of altering the genes of the embryo in the uterus is done, in some embodiments, by a process comprising the steps of:
A: preparing a pregnant animal carrying the embryo for at least one injection,
B: inserting a hollow device into a blood transport vessel of the pregnant animal, and
C: introducing genetic material into the blood transport vessel through the hollow device.

When using the technique as detailed in this specification, the genetic material was unexpectedly passed through the placenta, a known barrier to various materials and the genetic material was unexpectedly taken up and expressed (present) in all the cells of the embryo. Once present in all the cells, the effect of the alteration can be studied upon the embryo, the embryo's development, the animal after it is born, and the animal's offspring.

The disclosed technique therefore provides an inexpensive, rapid, efficient method to alter the genetic constitution of embryos and subsequently neonates and adult mice or other species of animal from those altered embryos. The experiments supporting this disclosure have shown that the transgene, or altered gene, is present in every cell of the transduced embryos and in the primordial germ cells that form sperm and eggs. This offers the ability to address genetic defects, or dim (decrease expression of) disease genes or to add missing genes, to model diseases, and to discover how genes function in both development and in birth defects.

As is evident to one of skill in the art, being able to target the gene in the developing embryo by injecting the genetic material of interest into the pregnant mother offers many opportunities. First, for many therapies, genes will need to be dimmed, not removed. One example is huntingtin, the gene that is mutated in Huntington's disease.

As huntingtin is expressed in every cell, it presumably has a function that has yet to be identified. Therefore, one would want to dim the expression of huntingtin, but not eliminate it entirely. By reducing its expression however, one could affect a therapeutic level that would not affect the cells in regions other than the brain.

Many birth defects are caused by what is called “gain of function”, a mutation that increases the expression or activity of a protein. By injecting the genetic material targeting that mutation into the pregnant mother, genetic alterations causing birth defects could be easily targeted prenatally (before tissue damage is done).

The technique disclosed in this specification has been demonstrated to target multiple genes using a single plasmid. Thus, in the case of Parkinson's disease, this technique could enable one to target, for example, both the precursor protein and the enzyme that degrades dopamine.

The technique can also be used to target multiple regions of a gene. This variation of the genetic material that is injected can be used when one is not sure which region of the gene is the most important.

The technique can be used to target multiple genes in a signaling pathway to understand their role in development or disease.

Researchers can also limit the alterations in the embryo by targeting genes in a particular tissue (e.g. the brain) by using a promoter that is expressed only in the nervous system. This technique could then be used to only treat the brain effects of diseases like Alzheimer's, while not affecting other tissues of the body.

This technique can also be used to more rapidly determine the therapeutic amount or length of treatments. For instance, developing constructs that can be turned on and off may be useful for the treatment of Parkinson's. For example, in the case of the fetal tissue implants in Parkinson's patients, they may make too much dopamine and cause side effects. By having controlled expression, one can monitor this and achieve a therapeutic level. Such controlled expression is rapidly obtained by using the technique disclosed in this specification.

This technique may also be used to alter the genes of animals which are otherwise difficult to make transgenic, otherwise known as “refractive to transgenesis”. The rat is one of these, since embryonic stem cells from rats have not been readily available.

The process used to alter the genes of the embryo, in some embodiments, comprised the steps of
A: preparing a pregnant animal carrying the embryo for at least one injection,
B: inserting a hollow device into a blood transport vessel of the pregnant animal, and
C: introducing genetic material into the blood transport vessel through the hollow device.

The method is believed to be much more effective when the blood transport vessel has been dilated prior to the introduction of the genetic material, and preferably prior to the insertion of the hollow device. Dilating the blood transport vessel can be achieved using techniques available to the practitioner which include the use of drugs or warming the pregnant animal. If drugs are used, care should be taken so as not to otherwise alter the development of the fetus. In some experiments, an alcohol swab over the tail produced vasodilation.

Warming the animal by exposing the animal to a temperature or combination of temperatures for a sufficient amount of time to dilate the blood vessel is a preferred method of dilating the blood vessel. In the case of CD-1 mice, the animals used in the experiments described herein, a warm blanket (approximately 10-20° C. above the temperature of the mouse's body temperature) was used. The mouse was laid on the blanket for about 5-10 minutes, or until the extremities became pink. Dilation of the tail veins were visually observed at this time.

It is further advantageous to immobilize the blood transport vessel of the pregnant animal. Examples of immobilization of the blood transport vessel of the pregnant animal are anesthetizing the pregnant animal, sufficiently securing the pregnant animal to permit insertion of the hollow device into the blood transport vessel; and the practitioner can have the pregnant animal itself immobilize the blood transport vessel (otherwise known as holding still). Animals which are cooperatively trainable may be able to do this. In humans, the practitioner could instruct the pregnant mother to hold still or not move the extremity containing the targeted blood transport vessel.

In the case of pregnant CD-1 (albino) mice or their neonates, they were placed into a small conical tube with a hole at both ends. The tail stuck out of one hole. While the tip of the tail could move, the tail vein at the base of tail was held virtually immobile so that the hollow device, in this case a needle, was inserted bevel up into the tail vein near the base of the tail.

While the hollow device used in these experiments was a needle, with the preferred needle being a butterfly needle, a shunt, or other similar device could also be used to pass the genetic material into the blood transport vessel.

The size of the opening of the needle relative to the rate of delivery is important to the success of technique. It was discovered that when using the high pressure teachings of earlier works (e.g. Lewis, et al, 2002), the mother died. For example, the technique used in Lewis is considered a “high pressure” technique whereas, the disclosed technique is a low pressure technique, with the DNA material or genetic material being introduced in a dilute solution over an extended period of time.

While the tail vein at the base of the tail was used in the experiments, it is contemplated that any blood transport vessel (artery or vein) can be used with the proviso that the artery or vein transports the blood from the point of insertion of hollow device, preferably a needle, to the embryo before reaching a capillary bed. Examples of suitable arteries and veins are uterine arteries which travel to the embryo and tail veins which travel to the embryo without reaching a capillary bed. In the case of animals without tails, such as humans, there are veins which are the functional equivalent of tail veins in mice. Generally, to be a functional equivalent of a tail vein, the vein should deliver the blood to the embryo before reaching the capillary bed of a heavily vascularized maternal organ or similarly functioning mechanism in the blood transport system. It is noted that the placenta and embryo are not maternal organs and is therefore the placenta is not a capillary bed to be avoided.

The time for introducing the genetic material is also an important consideration. The preferred time is after implantation of the embryo into a uterine wall and prior to establishment of the fetal circulation. Another preferred time for introduction is the time embryo begins gastrulation and prior to establishment of fetal circulation. This corresponds to the second half of the first trimester when the placental barrier is most permeable. For mice, it has been determined that the introduction of the genetic material is preferably done at about E6.0-E6.5 of embryonic development. Where E1 is day one, E2 is day two, and so on. Skilled artisan will appreciate that corresponding time frames in other species of animal.

The pressure at which the genetic material is introduced is called the “introduction pressure.” The introduction pressure is the pressure at the exit of the hollow device. The introduction pressure should be sufficiently low so as not to kill the pregnant animal. As demonstrated in the first set of experiments, pressures discussed in the art killed the pregnant mother.

Pressure at the exit side of the needle, or its functional equivalent should be less than the pressure which would kill the pregnant mother. In practical terms, this can be less than about the pressure required to pass 300 microliters of Ringer's solution at 23° C. through a 23 gauge needle in 10 seconds, or below about the pressure required to pass 300 microliters of Ringer's solution at 23° C. through a 23 gauge needle in 15 seconds or below about the pressure required to pass 300 microliters of Ringer's solution at 23° C. through a 23 gauge needle in 20 seconds or below about the pressure required to pass 300 microliters of Ringer's solution at 23° C. through a 23 gauge needle in 25 seconds, or below about the pressure required to pass 300 microliters of Ringer's solution at 23° C. through a 23 gauge needle in 30 seconds. Another variant is to introduce the genetic material through a drip into the blood transport vessel. This would be similar to an I.V. drip used to continuously administer drugs. Thus a catheter would work as the delivery device as well. In another embodiment, the introduction may be discontinuous, with the material being introduced on several occasions. If the pressure is too high, the mother will die.

If a needle is used, the pressure is usually generated by the plunger of a syringe. If a drip system like a catheter, the pressure is generated by keeping the storage device containing the genetic material above the hollow device entering the blood vessel.

Another method of introducing the genetic material is to use a carrier solution, with the genetic material preferably dissolved or suspended in the solution. The carrier solution should be of sufficient polarity to keep the genetic material unaggregated, as opposed to clumped or aggregated. The preferred distribution would be evenly distributed throughout the solution. Ringer's solution was used in the experiments and is an example of a suitable carrier.

The genetic material useful for this method is primarily naked DNA, which may be plasmid DNA (pDNA) not delivered in a viral vector. RNA variants are suitable as well, although the invention is not limited to these types of nucleic acid.

An exemplary DNA plasmid useful for this invention may be constructed as follows: While not essential to the genetic effect of the study target, the plasmid may contain a label such as a fluorescent or luminescent label, (e.g., DsRed2, EGFP-enhanced green fluorescent protein, and the like). This enables the practitioner to know which cells have the plasmid and which do not. Detection of the expression of the transgene in postnatal animals can be done by putting their ear or paw under UV light with the ones that carry the gene fluorescing, i.e., have green or red skin. A common technique is to insert neomycin resistance gene (so that the resulting cells become resistant to antibiotics), or any other gene that the practitioner would like to be co-expressed with the RNAi (or DNA).

The vector preferably has two constitutive promoters (that drive expression of the genes of interest); for example, one uses either CMV-(cytomegalovirus) (or ubiquitin) the other is H1 or U6 (RNA polymerase III promoters). While these were used to express the RNA, any other suitable gene could be inserted here too.

It is by these techniques that one knows that the primordial germ cells have had the target gene expressed as well. Because the experiments used a single vector (rather than using co-transfection) in which a DsRed2, or EGFP marker is expressed from the ubiquitin or CMV promoter, and the H1 or U6, RNA polymerase III promoter, drives expression of a hairpin RNAi targeted to the gene of interest resulting in constitutive expression of both the DsRed and the hairpin (Velkey and O'Shea, 2003). In the experiments used here, a 300 μl solution containing 10 μg plasmid DNA in the pRed vector to time (E6.5) pregnant females was used.

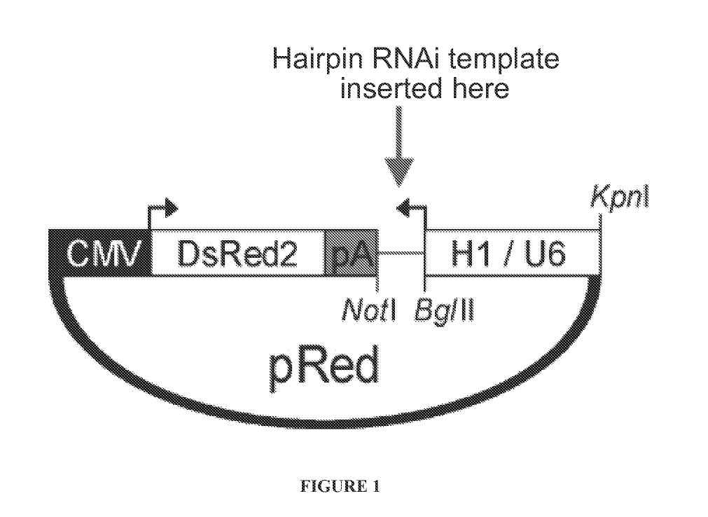

FIG. 1 is but one vector used in the experiments. The naked DNA as shown in FIG. 1 contains a construct in which a promoter driving the expression of a fluorescent gene, such as DsRed or GFP; hairpin RNA (The RNAi Template) which targeted the RNA responsible for the expression of the gene of interest. Because this construct is not in a viral delivery system, it is known as naked DNA. Therefore, it was naked DNA which was the genetic material introduced into the blood transport vessel, (e.g., the tail vein).

The technique to make the vector is well known. For example, the plasmid can be constructed as described in Velkey and O'Shea, “Oct4 RNA Intereference Induces Trophectoderm Differentiation in Mouse Embryonic Stem Cells”, genesis 37:18-24, 2003, the teachings of which are herein incorporated in its entirety.

The amount of DNA is up to the practitioner and may be modified for the particular application.

In the first set of experiments, the vector was DNA containing the promoter CMV—(cytomegalovirus) driving the expression of DsRed2 and a second promoter, U6, driving the expression of the hairpin DNA targeted to knockdown the expression of Bmp4. In this set of experiments, all the cells of the embryos, including the primordial germ cells, fluoresced indicating that the construct was taken up and expressed in those cells, which included the primordial germ cells. The chance of a false positive, where the marker is expressed, but the hairpin DNA is not, was eliminated by placing the marker and the DNA on the same backbone.

Because the hairpin DNA was on the same backbone as the marker and all the cells had the marker, there was no reason to believe that the Bmp4 had not been knocked down as well. This predictability has been confirmed over the course of 9 separate DNAs. In each case, using the disclosed technique, the marker was found in all cells of the embryo or neonate born from the embryo. In all cases, the expression of the naked DNA introduced into the tail vein was individually confirmed at the protein and RNA level.

In the case of the first experiments, the knockdown of Bmp4 was individually confirmed at the protein and RNA levels using immunohistochemistry and RT-PCR (reverse transcript, polymerase chain reaction).

Other experiments using the method described above under the conditions described show the effectiveness of the method to alter the gene expression of the embryo are Bmp4 (which, to the extent it can be analyzed, phenocopies the Bmp4 null embryos), Bmp7 alone, Bmp4 in combination with Bmp7, Wnt8a, Wnt8b (singly and in combination) (where Wnts are mammalian homologues of wingless genes identified in the fruitfly), Nanog, geminin, Est1, Est2, and Est3, where EST is an Expressed Sequence Tag (EST); which are genes (without names) that the inventors identified in a screen for genes whose expression in embryonic stem cells were unregulated by exposure to noggin; i.e., new genes.

In each case where an antibody is available to the protein (Bmp4, Nanog, geminin) or to the downstream signal transduction cascade (PhosphoSmad1,5,8) knock-down of the gene product was observed in individual embryos. In cases where an antibody is not available, we have demonstrated unique phenotypes, and knock-down by PCR (polymerase chain reaction).

The data demonstrates therefore that multiple targets can be knocked-down or reduced—e.g., Bmp7 and Bmp4 and identify an additive phenotype.

Scrambled (missense) hairpin DNA exposed embryos have been produced that resemble wild type and pRed embryos both in phenotype and gene expression profile. Multiple genes using a single construct (noggin, chordin and follistatin) and confirmed knock-down using quantitative PCR on individual embryos.

In every instance where this method has been used, the marker and gene have been expressed in all the cells of the embryo or postnatal mouse, including the primordial germ cells.

We claim:

1. A method for altering gene expression in an embryo, comprising: a) providing a pregnant animal carrying an embryo; and b) administering nucleic acid to said pregnant animal under conditions that permit said nucleic acid to be transferred from said pregnant animal to said embryo and wherein said nucleic acid is biologically active in said embryo.

2. A method to modify an embryo comprising the steps of:
A: preparing a pregnant animal carrying the embryo for at least one injection, wherein the step of preparing the pregnant animal comprises at least one step selected from the group consisting of: dilating a blood transport vessel of the pregnant animal, warming the pregnant animal, immobilizing the blood transport vessel of the pregnant animal, anesthetizing the pregnant animal, sufficiently securing the pregnant animal to permit insertion a hollow device into the blood transport vessel, and having the pregnant animal immobilize the blood transport vessel;
B: inserting a hollow device into the blood transport vessel of the pregnant animal;
wherein the blood transport vessel transports blood from the point of insertion of the hollow device to the embryo avoiding the capillary bed of a heavily vascularized maternal organ,
C: introducing genetic material into the blood transport vessel through the hollow device,
wherein the introduction of the genetic material is done after implantation of the embryo into a uterine wall and prior to establishment of fetal circulation in the embryo;
wherein, the introduction of the genetic material is done at an introduction pressure sufficiently low so as not to kill the pregnant animal.

3. The method of claim 2, wherein said hollow device is selected from the group consisting of a needle, a shunt, and a catheter.

4. The method of claim 2, wherein said blood transport vessel is selected from the group consisting of uterine arteries, tail veins and the functional equivalent of tail veins in animals without tails.

5. The method of claim 2, wherein said genetic material is present in a carrier solution.

6. The method of claim 2, wherein said genetic material is selected from the group consisting of DNA, naked DNA, plasmid DNA, and RNA.

7. A method of modifying an embryo comprising the steps of:
A: preparing a pregnant animal carrying the embryo for at least one injection,
B: inserting a hollow device into a blood transport vessel of the pregnant animal, and
C: introducing genetic material into the blood transport vessel through the hollow device so as to permit transfer of said genetic material to said embryo.

8. The method of claim 7 wherein the step of preparing the pregnant animal to receive the at least one injection further comprises the step of dilating a blood transport vessel.

9. The method of claim 8 wherein the step of dilating the blood transport vessel is done by warming the pregnant animal.

10. The method of any of claim 7 wherein the step of preparing the pregnant animal comprises immobilizing a blood transport vessel of the pregnant animal.

11. The method of claim 10 wherein the process of immobilizing the blood transport vessel of the pregnant animal is selected from the group consisting of anesthetizing the pregnant animal, sufficiently securing the pregnant animal to permit insertion of the hollow device into the blood transport vessel, and having the pregnant animal immobilize the blood transport vessel.

12. The method of any of claim 7 wherein the hollow device is selected from the group consisting of a needle and shunt.

13. The method of any of claim 7 wherein the hollow device is a needle.

14. The method of claim 13 wherein the needle is a butterfly needle.

15. The method of claim 13 wherein the needle is a 23 gauge needle.

16. The method of claim 8 wherein the blood transport vessel is selected from the group consisting of blood transport vessels which transport blood from the point of insertion of the hollow device to the embryo before reaching the capillary bed of a richly vascularized maternal organ.

17. The method of claim 8 wherein the blood transport vessel is selected from the group consisting of uterine arteries, tail veins and the functional equivalent of tail veins in animals without tails.

18. The method of claim 7 wherein the introduction of the genetic material is done after implantation of the embryo into a uterine wall and prior to establishment of fetal circulation in the embryo.

19. The method of claim 7 wherein the introduction of the genetic material is done after the embryo is at the early egg cylinder stage of development and prior to establishment of fetal circulation.

20. The method of claim 7 wherein the pregnant animal is a mouse and the introduction of the genetic material is done at about E6.0-E6.5 of the embryo's development stage.

21. The method of claim 7 wherein the introduction of the genetic material is done at an introduction pressure, wherein said introduction pressure is sufficiently low so as not to kill the pregnant animal.

22. The method of claim 7 wherein the introduction of the genetic material is present in a carrier solution.

23. The method of claim 22 wherein the carrier solution is of sufficient polarity to keep the genetic material evenly distributed throughout the carrier solution.

24. The method of claim 23 wherein the carrier solution is Ringer's solution.

25. The method of claim 7 wherein the genetic material is selected from the group consisting of DNA, naked DNA, and RNA.

26. The method of claim 21 wherein the introduction pressure is below about the pressure required to pass 300 microliters of Ringer's solution at 23° C. through a 23 gauge needle in 10 seconds.

27. The method of claim 26 wherein the introduction pressure is below about the pressure required to pass 300 microliters of Ringer's solution at 23° C. through a 23 gauge needle in 15 seconds.

28. The method of claim 27 wherein the introduction pressure is below about the pressure required to pass 300 microliters of Ringer's solution at 23° C. through a 23 gauge needle in 20 seconds.

29. The method of claim 28 wherein the introduction pressure is below about the pressure required to pass 300 microliters of Ringer's solution at 23° C. through a 23 gauge needle in 25 seconds.

30. The method of claim 29 wherein the introduction pressure is below about the pressure required to pass 300 microliters of Ringer's solution at 23° C. through a 23 gauge needle in 30 seconds.

31. The method of claim 26 wherein the introduction of the genetic material is done through a drip into the blood transport vessel.

32. The method of claim 26 wherein the introduction of the genetic material is done in a discontinuous manner.

33. A process to modify the genes of an embryo comprising the steps of:
A: preparing a pregnant animal carrying the embryo for at least one injection, wherein the step of preparing the pregnant animal contains at least one step selected from the group consisting of dilating a blood transport vessel of the pregnant animal, warming the pregnant animal, immobilizing the blood transport vessel of the pregnant animal, anesthetizing the pregnant animal, sufficiently securing the pregnant animal to permit insertion a hollow device into the blood transport vessel, and having the pregnant animal immobilize the blood transport vessel;
B: inserting a hollow device into the blood transport vessel of the pregnant animal, wherein the hollow device is selected from the group consisting of a needle, shunt, catheter, butterfly needle, and a 23 gauge needle;
wherein the blood transport vessel transports blood from the point of insertion of the hollow device to the embryo avoiding the capillary bed of a heavily vascularized maternal organ and may optionally be selected from the group consisting of uterine arteries, tail veins and the functional equivalent of tail veins in animals without tails,
C: introducing genetic material into the blood transport vessel through the hollow device,
wherein the introduction of the genetic material is done after implantation of the embryo into a uterine wall and prior to establishment of fetal circulation in the embryo
wherein, the introduction of the genetic material is done at an introduction pressure sufficiently low so as not to kill the pregnant animal and wherein the introduction of the genetic material is optionally present in a carrier solution and wherein the genetic material is selected from the group DNA, naked DNA, plasmid DNA and all the variants of RNA.
